(19) United States
(12) Patent Application Publication
(10) Pub. No.: US 2008/0216842 A1
(43) Pub. Date: Sep. 11, 2008

(54) Contraceptive device designed for mexican women according to the dimensions of their uterine cavity acoording to their parity

(76) Inventor: Guillermo Sanders Acedo, Distrito Federal (MX)

Correspondence Address: DEFILLO & ASSOCIATES, INC., P.O. Box 14104, Clearwater, FL, 33766, US

(21) Appl. No.: 12/070,476

(22) Filed: Feb. 19, 2008

Related U.S. Application Data
(63) Continuation-in-part of application No. 10/580,328, filed on May 24, 2006.

(51) Int. Cl. A61F 6/14

(52) U.S. Cl. 128/839

(57) ABSTRACT

The present invention relates to a flexible plastic intrauterine device with contraceptive aim, which has a “T” shape. The length of the horizontal arms coincides approximately with the width of the uterine bottom according to the parity and the length of the vertical rod of the frame with the functional uterine cavity of the nulligest, nulliparous, young and multiparous Mexican women. The intrauterine device is presented in five models whose measurements of length of the frame or of the tilde or horizontal arms are from 13 to 33 mm and the length of the post or vertical rod is from 21 mm to 39 mm. Attached or injected thereto is included a plastic monofilament from 10 to 40 cm in length in the distal end of the post or vertical rod whose shape is a spherical, hexagonal or flat. The plastic frame of medical grade contains wound in the tilde or both horizontal arms of the “T” a copper filament from 0.24 to 0.26 mm in diameter or a copper tube from 2.1 to 2.2 mm in exterior diameter, and a length of 4.9 to 5.2 mm that is 99.99% pure. Furthermore in the post or vertical rod there will also be a winding of copper filament between 0.24 to 0.26 mm in diameter, whose total surface, of the horizontal arms and of the post or vertical rod is between 200 and 380 mm2. Plastic frame of radiopacifiant material of 23%±5% and 77%±5% of flexible medical plastic grade and viscoelastic or deformation properties not greater than 5 mm of its original form.

### RELATED U.S. APPLICATION

This application is a continuation-in-part of Ser. No. 10/580,328 filed May 24, 2006, entitled “METHOD FOR THE PRODUCTION AND POSITIONING OF AN INTRAUTERINE DEVICE WHICH IS ADAPTED TO THE UTERINE CAVITY MEASUREMENTS,” pending, the disclosure of which is incorporated herein by reference.

### FIELD OF THE INVENTION

This invention has the fundamental intention to substantially diminish the collateral effects, mainly bleeding and pain, of contraceptives which are constituted by a plastic frame in form of a T and medical grade copper, by means of its adaptation to the measurements of the uterine cavity of Mexican women.

### BACKGROUND OF THE INVENTION

In the decade of the Sixties and early Seventies, a signal arose as world-wide alarm, produced by the population increase that threatened to overflow the natural reproductive limits that during thousands of years humanity had enjoyed. This phenomenon was located, mainly, in the developing countries thereby increasing the pressure on their natural resources, environment and putting into risk their modest economic advances achieved until then.

Under these circumstances there appeared the first modern contraceptives: the synthetic hormones in their diverse presentations: oral, injectable, cutaneous, subcutaneous. Intrauterine dispensers and, to a smaller grade, salpingo-ovariectomy appears as well, also the different models of contraceptive intrauterine devices, the copper T among them, which achieved and still conserves justly a great acceptance among the user population¹.

Nevertheless, in spite of the considerable advantages and effectiveness of this contraceptive, an important number of users showed from the beginning complaints and rejections through annoyances like: pains; colics; bleedings; syncopes and spontaneous expulsion²³⁴.

It was evident that the dimensions of the intrauterine devices did not correspond to the uterine measurements of the users. They were too large.

Under these circumstances smaller devices were designed: 19.0 mm wide by 29.4 mm in length for nulliparous which represented the opposite end. That is to say, nobody seemed to realise that the uterine cavities of the women do not only modify their characteristics and dimensions during menstruation, but fundamentally they do due to parity and ethnic group. Numerous investigations were made which, while advancing, verified the justified complaints of the users in the different countries and continents.

The universal acceptance of Intrauterine Device Copper T 380 stirred up the development of diverse forms and models with different amounts of copper that except for a couple of exceptions did not consider the recommendations contained in a lengthy and thorough study sponsored by the WHO; The UN; World Bank; and other Institutions to take advantage of the unquestionable contraceptive qualities of the IUD but intending to diminish their main collateral effects: bleeding; pain, with which the acceptance and continuity of the method could be increased by 25% to 30%⁵. These annoyances, bleeding and pain were reported simultaneously with the introduction of the IUD and have been documented since then.

Effectively the publication of the Population Council in 1992, a group of prestigious American investigators headed by Irving Sivin summarize among other many subjects the undesirable collateral effects: bleeding and pain as well as pelvic infection, indicating that this latter risk is higher right after the insertion.

In fact, in investigations in the U.S.A itself that already go back to 1976 Dr. Hasson warned about the disadvantages of the method⁶.

In Germany Dr Kurz, points out the same phenomenon through investigations published in 1981 and 1984⁷⁻⁸

The same collateral effects were studied in Sweden⁹; Canada¹⁰; Spain¹¹ and in México¹²⁻¹³ among many other nations, which essentially match each other, and it should be emphasized that the study by W. R. Hohman of the U.S.A. by means of an electron microscope determined the pressure generated between the IUD and the uterus as the cause of the abnormal bleeding¹⁴.

On the other hand there have been cases of pelvic infection (“PID” by its abbreviation in English) associated to the presence of the IUD which, if not taken care of adequately, may cause infertility.

Also, about this medical incident numerous studies and investigations exist which widely and deeply approach this risk. Outstanding is, among many others, the one of Catherine Richey titled: New attention to the IUD published in Population Reports¹⁵ which shows that “ . . . with the IUD the infection risk is very low”. On the other Hand Dr Hubacher in his investigatión¹⁶ as well as in his “point of view” does a documented historical revision on the future of the IUD, pointing out among other things, a picture showing 14 studies in the last 15 years that the PID and probable infertility are originated or produced by the bacteria C. trachomatis, and not by the intrauterine Copper T device¹⁷.

And for further knowledge, the study entitled “Intrauterine Device and Pelvic Inflammatory Disease (PID), an International Perspective by Timothy A. M. Farley et al.” published in The Lancet in 1992 was presented, which affirms that the pelvic infection referred to the process of insertion as well as to the antecedents of transmission risk of venereal diseases¹⁸.

However, these complaints were not heeded, although in Germany, Mexico, the United States and other countries, clinical studies were made, in which the same cavimetries took place that demonstrated the disproportion between the uterine cavity and the available devices, all works that date from 1973, 1979, 1982, 1984.¹⁹⁻²⁰⁻²¹⁻²²

In the following table the results obtained by Mexican investigators of the cavimetries appear in their two published works:

TABLE 1

| | | | | | width = −1 | length = −1 |
|---|---|---|---|---|---|---|
| | | Standard | Average | Standard | Standard | Standard |
| Parity | Average width | deviation | length | deviation | Dev. | Dev. |
| 0 | 24.3 | 6.9 | 34.8 | 6.4 | 17.4 | 28.4 |
| 1 | 25.9 | 5.8 | 36.0 | 6.1 | 20.1 | 29.9 |
| 2 | 26.6 | 5.6 | 38.5 | 6.6 | 21.0 | 31.9 |
| 3 | 25.5 | 5.7 | 39.0 | 6.3 | 19.8 | 32.7 |
| 4 | 27.6 | 6.8 | 37.8 | 6.8 | 20.8 | 31.0 |
| 5 | 27.1 | 5.7 | 39.8 | 7.6 | 21.4 | 32.2 |

Dimensioning: mm

Therefore the manufacturing of a variety of contraceptive models is essential, which, while preserving their recognised advantages adapt to the measurements of the uterine cavity of nulligests, nulliparous and multiparous Mexican women. As it can be seen in tables 1 and 2, the disproportion between the dimensions of the uterine cavity and the devices is resolved by means of the manufacturing of five models of intrauterine devices with copper, the same that correspond to the measurements of the uterine cavity of mexican women, for maximum range, that were registered by mexican investigators.

### BRIEF DESCRIPTION OF THE DRAWINGS

FIG. 1 ILLUSTRATES the intrauterine device having a frame in form of a “T” according to the present invention.

FIG. 2 illustrates the uterine cavity in which the intrauterine device of FIG. 1 can be observed.

FIG. 3 illustrates the plastic frame for parity 00 (nulligests or those who never have had a pregnancy).

FIG. 4 illustrates the plastic frame for parity 0 (who had not a childbirth previously).

FIG. 5 illustrates the plastic frame for the parity 1 (who has had one childbirth).

FIG. 6 illustrates the plastic frame for parity 2 (with two previous childbirths).

FIG. 7 illustrates the plastic frame for parity 3 (Three previous childbirths).

FIG. 8 illustrates the mountable applicator and the insertion tube with the stopper or cervical spacer of the present invention.

FIG. 9 illustrates the insertion tube, stopper or cervical spacer, and the mountable applicator according to the present invention.

### DESCRIPTION OF THE INVENTION

The intrauterine devices are characterized in that they have a flexible T-shaped plastic frame that has in its post or vertical rod copper filament and in its tilde or horizontal arms copper filament and/or copper tubes. They must be made of medical grade plastic material preferably being inert like polypropylene, polyethylene, nylon, dacron, silastic, etc. And the copper with a purity degree of 99.99%, whose post or vertical rod and tilde or horizontal arms surface is of between 200 and 380 mm².

The present invention satisfies the necessity of Mexican women to have an effective, safe and reversible contraceptive according to the measurements of their uterine cavity. Therefore, the following models appear characterized by having a form of a “T”.

TABLE 2

| WIDTH | | | | LENGTH | | | |
|---|---|---|---|---|---|---|---|
| Parity | Minimum* | average | maximum** | Parity | Minimum* | average | maximum** |
| Nulligest | | | | Nulligest | | | |
| 00 | 13 mm | 18 mm | 23 mm | 00 | 21 mm | 26 mm | 31 mm |
| Nulliparous | | | | Nulliparous | | | |
| 0 | 17 mm | 22 mm | 27 mm | 0 | 23 mm | 28 mm | 33 mm |
| for 1 | 19 mm | 24 mm | 29 mm | For 1 | 25 mm | 30 mm | 35 mm |
| for 2 | 21 mm | 26 mm | 31 mm | for 2 | 27 mm | 32 mm | 37 mm |
| for 3 | 23 mm | 28 mm | 33 mm | for 3 | 29 mm | 34 mm | 39 mm |

*minus 5 mm
**plus 5 mm

The previous measurements are taken from the measurements of the uterine cavities of Mexican women, showed in Picture 1 and fitting them with the intention of obtaining the maximum range, thereto incorporating the nulligest women. These two measurements will be able to adjust themselves to one or two millimetres more or less in order to consider the contractibility of the materials used, as well as the variation of the measurements of the uterine cavity during menstruation. The length of the post or vertical rod has a reduction of 5 mm to avoid it penetrating into the internal ostium of the uterine neck²³.

In any case, the measurements that this invention protects are the indicated ones in table no. 2. This invention is better understood by observing the following figures:

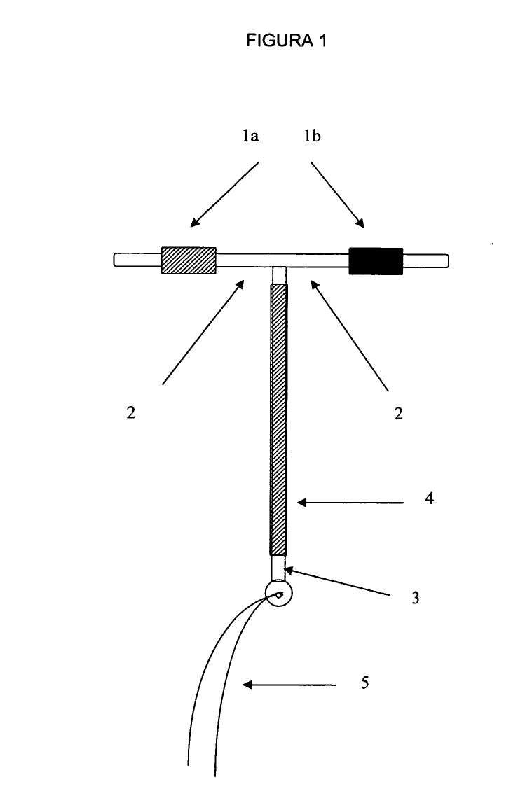

FIG. 1 represents a scheme of the intrauterine device characterized by having a frame in form of a “T”, in whose tilde or horizontal arms there is indifferently copper filaments or copper tubes contained:
1. Copper filament or copper Tube,
2. the tilde or horizontal arms of the plastic frame with copper wire winding and/or copper tube.
3. Post or vertical piston rod of the plastic frame
4. Copper filament winding.
5. Monofilament of plastic.

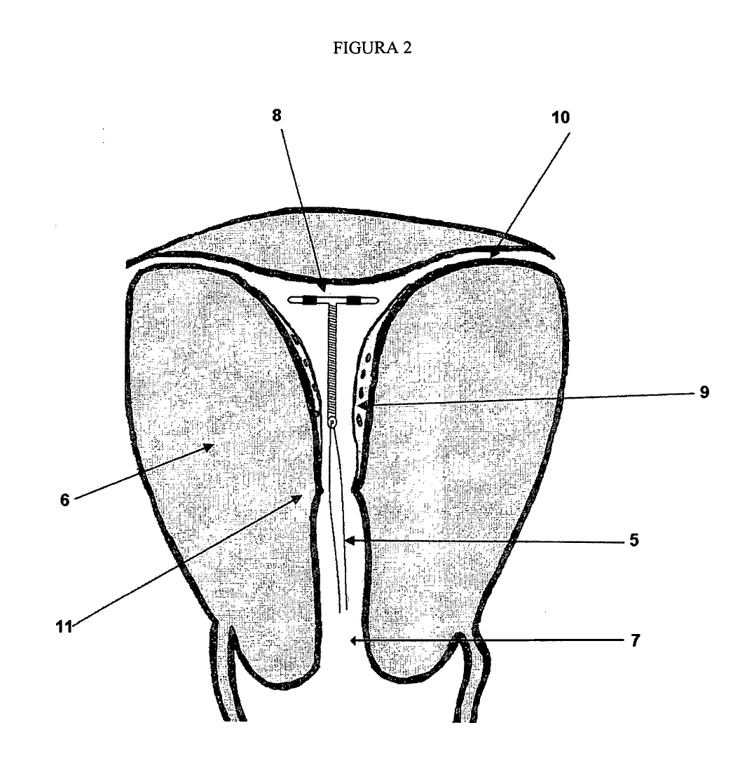

FIG. 2: It represents a diagram of a longitudinal section of the uterine cavity in which the position of the T-shaped intrauterine device can be observed.
6. Monofilament of plastic
7. Uterine body
8. Cervical channel.
9. Uterine bottom.
10. Endometrium.
11. Oviducts 11
12. Internal Ostium

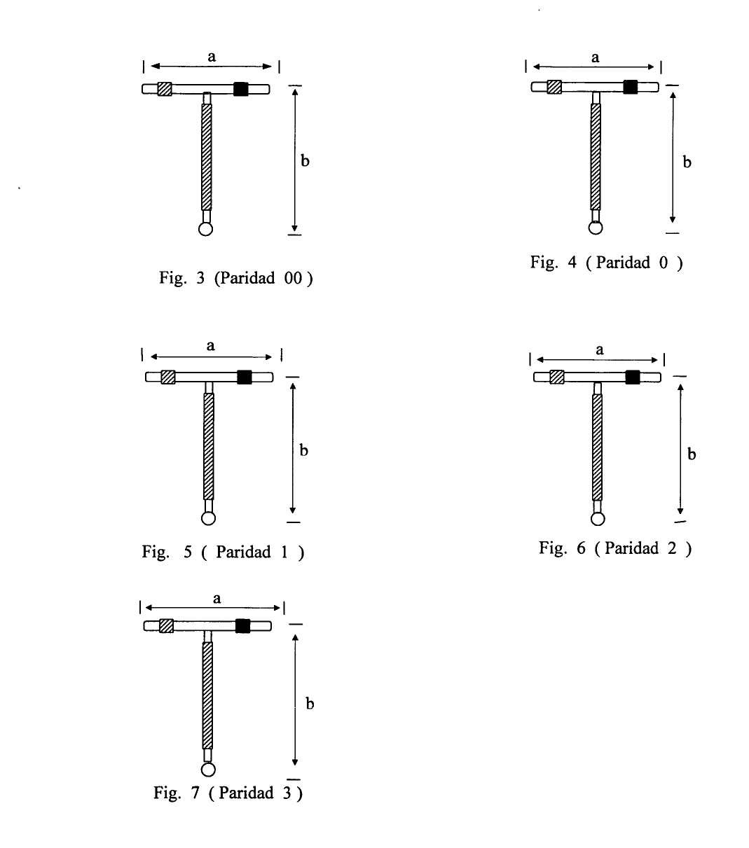

FIG. 3 shows the plastic frame for parity 00 (nulligests or those who never have had a pregnancy) with the following measurements:

a) Width of the tilde or horizontal arms of 13 mm to 23 mm; average 18 mm
b) Length of the post or vertical piston rod of 21 mm to 31 mm; average 26 mm.
c) Copper surface: 200 to 380 mm2 distributed along the tilde or horizontal arms
and the post or vertical rod of the T-shaped plastic frame.

FIG. 4 shows the plastic frame for parity 0 (who had not a childbirth previously) with the following measurements:

Width of the tilde or horizontal arm of 17 mm to 27 mm; average 22 mm.
Length of the post or vertical rod of 23 mm to 33 mm; average 28 mm.
copper Surface: 200 to 380 mm2 distributed along the tilde or horizontal arms and the post or vertical rod of the T-shaped plastic frame.

FIG. 5 shows the plastic frame for the parity 1 (who has had one childbirth) with the following measurements:

Width of the horizontal arm: of 19 mm to 29 mm; average 24 mm.
Length of the post or vertical rod of 25 mm to 35 mm; average 30 mm.
Copper surface: 200 to 380 mm2 distributed along the tilde or horizontal arms and the vertical rod of the T-shaped plastic frame.

FIG. 6 shows the plastic frame for parity 2 (with two previous childbirths) with the following measurements:

a) Width of tilde or horizontal arms: of 21 mm to 31 mm; average 26 mm.
b) Length of the post or vertical rod: of 27 mm to 37 mm; average 32 mm.
c) Copper Surface: 200 to 380 mm2 distributed along the tilde or horizontal arms and the post or vertical rod of the T-shaped plastic frame.

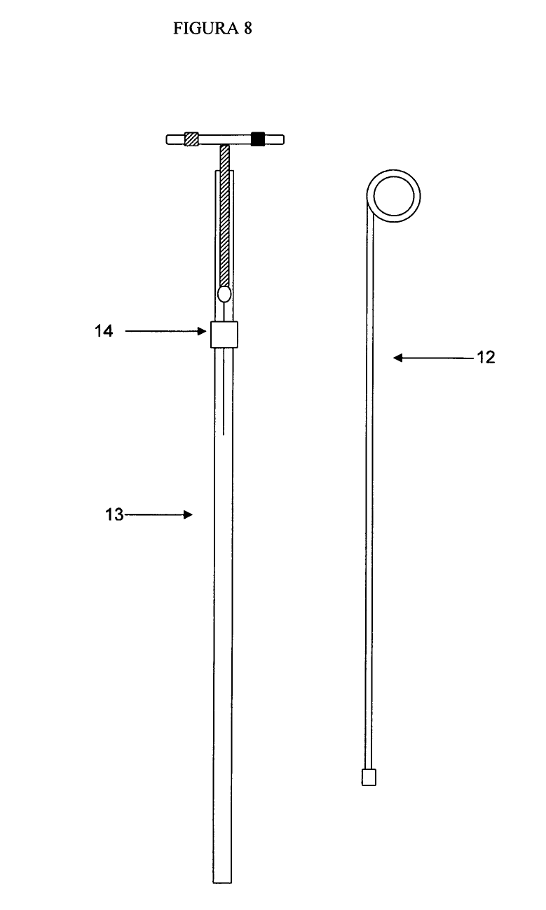

FIG. 7 shows the plastic frame for parity 3 (Three previous childbirths) with the following measurements:

a) Width of the tilde or horizontal arms of 23 mm to 33 mm; average 28 mm.
b) length of the post or vertical rod of 29 mm to 39 mm; average 34 mm.
c) copper surface: 200 to 380 mm2 distributed along the tilde or horizontal arms and the post or vertical rod of the T-shaped plastic frame.

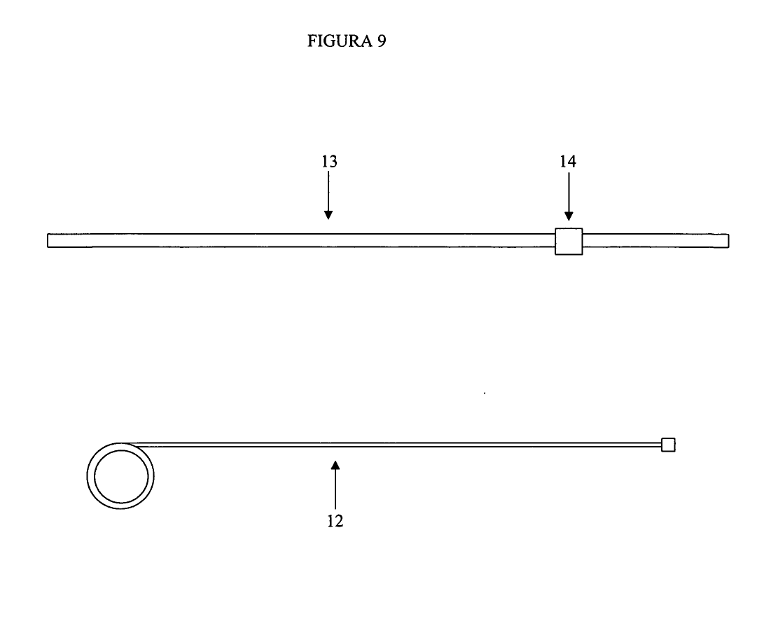

FIG. 8 shows the mountable applicator (12) and the insertion tube (13) with the stopper or cervical spacer (14) of the present invention, with the device being ready for its positioning in the uterine cavity.

FIG. 9 shows the insertion tube (12), stopper or cervical spacer (13) and the mountable applicator (14).

In FIG. 2 there stands out the plastic monofilament (5) within the cervical channel (7) of the vagina.

As having been described previously, the intrauterine device of the present invention has the form of a “T” as shown in FIGS. 3 to 7. The measurements of the tilde or horizontal arms (width) are between 13 and 37 mm and the length of the post or vertical rod of 21 to 41 mm for the five models. The section diameter of the device is typically within the range of 1 to 4 mm. The length of the post or vertical rod (3) and in the tilde or horizontal arms (2) of FIG. 1, is arranged such that the device complies within the uterine cavity as shown in FIG. 2 so that the tilde or horizontal arms extend on the uterine bottom (8) without penetrating the oviducts nor pressing the sensitive areas where the childbirth (10) contractions of the FIG. 2 begin, and the post or vertical arm of the device (3) of FIG. 1, complies throughout the uterine cavity, without penetrating in the cervical channel (7) of FIG. 2.

### Action Mechanism²⁴

Although the present invention is not limited by any theory and its action mechanism, based on studies and investigations that have been made in diverse countries, the intrauterine device related to the present invention is considered to be acting in several ways, mechanical, biological, biochemical and biological.

Mechanically, when acting on the uterine walls it causes a peristaltic movement; biochemically, it generates an inflammatory aseptic reaction of rejection that modifies the characteristics of the endometrium and of the moist environment of the uterine cavity, and biologically in the balance of the endometrial prostaglandins, increase of oedema and the vascular permeability with infiltration of leukocytes, including neutrophils, mononuclear cells and macrophages.

All the previous produces hostile means for the migration of the spermatozoa through the uterine cavity, in addition, the copper ions generated by the device increase the inhibiting effect of the motility of the spermatozoa, as much in the uterine cavity as in the cervical mucosa. This could be demonstrated in studies for recovering of spermatozoa of the peritoneal cavity or different segments from the feminine genital tract after natural or artificial insemination during the fertile period of users of intrauterine devices, being absent or present in a very small amount in the higher part of the feminine genital tract, particularly in the case of the devices with copper.

Normally, the human hormone chorionic gonadotrophin (HGCH) can be detected some days after the fertilization of the ovum. In recent specific studies of immunoradiometric test for the terminal peptide of the beta chain of the HGCH, that has demonstrated to be more specific than other procedures of radio-immuno-test, they have not been able to demonstrate an increase in the levels of this hormone, which suggests that the copper devices, more than to interfere with the nidation of the ovum, they have truly acted like contraceptives and not like microabortifacients.

Typically the device of the present invention is of such size that the portion of the tilde or horizontal arms of the “T” extends so that its presence generates a reaction of strange body. The X-ray studies indicate that the ends of the tilde or horizontal arms come to be near the endometrium (9) anchoring to that level of FIG. 2. Nevertheless it has been considered that by reducing the diameter of section, the lateral and antero-posterior pressure on the uterus walls is reduced.

As shown in FIG. 2 the uterine body (6) when being normally relaxed causes a virtual obliteration of the uterine cavity. Although it is always mentioned as a cavity, in the relaxed position of the uterine muscle it contains a minimum or null amount of liquid, since the uterine walls are collapsed one on the other. It is a consequence of this relaxation state of the uterus that the intrauterine devices adapt by applying a certain antero-posterior and lateral pressure of the uterine muscle, so that they adapt to the walls of the uterine cavity. It is considered that this pressure generates an involuntary reaction that is the cause of some cases of expulsion, bleeding or pain. Due to the foregoing, as shown in FIG. 2, the intrauterine device of the present invention essentially adapts to the form and dimensions of the uterine cavity of the nulligests, nulliparous and young and/or tiny women and women with a childbirth or more, in its state of relaxation of way to specially apply single a minimum pressure to the walls to the endometrial mucosa.

As shown in FIGS. 8 and 9, the insertion tube (13) of the present invention has a length such that it allows it to extend itself through the interior of the uterine cavity up to the uterine neck and to extend in the vagina to the exterior to allow the doctor to be able to manipulate easily the whole set.

The outer diameter of the insertion tube (13) of FIG. 8, of between 3.4 and 5.4 mm facilitates the insertion of the intrauterine device in nulligests and nulliparous normally showing a narrow endocervical channel (multiparous usually have it wider). The easy insertion through the conduit of the uterine neck within the uterine cavity is carried out in agreement with the measurements conducted to the dimensions of the uterus, by adjusting the stopper (14) on the insertion tube (13) of FIG. 9, using the rule printed on the pattern.

The insertion tube loaded with the intrauterine device (IUD) is introduced (FIG. 8) through the cervical channel (7) of FIG. 2, by means of the system of “pushing” taking care of not touching the vaginal walls, until the stopper (14) of FIG. 9, makes contact with the uterine neck and the “T” touches the bottom of the uterus (8) of FIG. 2.

It is necessary to indicate that the small diameters of the tilde or horizontal arms including winding of the copper wire or the copper tube, and the small diameter of the insertion tube are fundamental factors in nulligest, nulliparous and multiparous women, since the risk of syncope, bleedings, pain and spontaneous expulsion is reduced, also the perforation probabilities diminish.

Once introduced up to the uterine bottom the device is released within the uterine cavity when retiring smoothly the insertion tube on the applicator (12) of FIG. 9, the applicator that is introduced in the insertion tube, which allows that the device given its own memory and elasticity recovers its original form adapting to the uterine cavity in natural form. The insertion tube (13) and the applicator (12) can be observed in FIG. 8.

In order to facilitate the extraction of the intrauterine device and to verify the permanence of the same, in the distal end of the post or vertical rod there is added a plastic monofilament (5) of FIG. 1, of between 10 and 40 cm. in length and a diameter between 0.20 to 0.30 mm for its use in the interval and post-childbirth or immediate post-abortion in young women or multiparous with contraception aims. The doctor cuts monofilaments to the desired length (extending out of the uterine neck 3 or 4 cm within the vagina). This monofilament (5) is shown in FIG. 2.

### Expulsion of the Devices

Clinical studies have been made to determine the ranges of expulsion, annoyances and pregnancies of the T-shaped intrauterine device similar to the one of the present invention in comparison to other typical intrauterine plastic devices that has been used previously, determining that these events are produced by the dimensional mutual incompatibility of the uterine cavity and the present devices.

### Abandonment of the Method

The consequences derived from placing devices that are too large or small in the uterine cavity, cause, since repeatedly demonstrated, collateral effects and similar consequences; abandonment of the method, undesired pregnancies and abortions. 46% of the Mexican women who accept the intrauterine device as contraceptive method do not continue with it²⁵. For that reason, in the present invention the five measurements shown in picture 2 are proposed.

### Effectiveness

The rate of permanence within the programs of familiar planning is very superior and the rate of effectiveness is increased in direct proportion with the copper surface without exceeding the 380 mm2 of the device, according to what can be seen in the following picture²⁶

PEARL INDEX (PREGNANCIES PER 100 WOMEN A YEAR)
“T”-SHAPED DEVICES ACCORDING TO THE QUANTITY
OF COPPER
PEARL-INDEX

| | |
|---|---|
| T - 200 | 2.5 |
| T - 220 | 0.9 |
| T - 380 | 0.5 |

### Social and Economic Advantages:

The main cause of death in women with ages between the ages of 14 and 45 years is an obstetric one.

In our country there take place between 800,000 and 1,500,000 annual abortions and their rate of mortality is very high, it is estimated that 1500 women die annually.

Also, the main problems of morbidity in women are related to pregnancy and childbirth. And it is in the adolescence where morbidity rates of and perinatal mortality appear to be the highest.

Independently of the previous, the barriers constituted by the insufficient medical infrastructure, human and economic resources, distances and communications, prevent that other products are practical in their application on a great scale. The National Council of Population, has identified in our country 201,000 localities, and around 170,000 have, each one, less than 500 inhabitants.

Intrauterine device T 380 has the advantage to be able to be economically included into institutional programs at large scale and to not require any training or special care on user side. It does not need any special discipline to follow a certain regime.

On the other hand, unlike hormonal, oral or injectable contraceptives that for many years requires regularity in its use, be it or daily or monthly, to guarantee their effectiveness, the devices, once inserted, maintain their contraceptive effectiveness.

The existence of intrauterine devices specially designed for Mexican nulligest women, nulliparous and multiparous fills a gap in the necessities of the Family Planning Programs, because it allows that an enormous group of women specially exposed have a comfortable method to surely and effectively space and slow down their pregnancies.

The present invention is a substantial improvement to the present devices, not only due to their better design for adapting into the uterine cavity, which is subjected to variations by seeing itself subjected or not childbirths during the fertile life of the women, but to the presence of the filament or copper tubes, medical grade, with a contact surface in such amount that increases the chemical-ionic effect that prevents the migration of the spermatozoa through the uterine cavity, an amount of copper that must be there according to the fecundity stage, which theoretically diminishes in to a greater extent the possibility of fertilization of the ovum, and this is obtained within the parameters indicated in the body of this document, by conserving form, elasticity and the general characteristics of the intrauterine devices that have demonstrated to be most effective.

Having described the intrauterine devices for the Mexican women: nulligests, nulliparaous or multiparous call to each other like property:

1. A intrauterine contraceptive device for Mexican women according to the measurements of the uterine cavity according to its parity comprising:
a flexible T-shaped plastic frame including horizontal arms having a cylindrical form of blunt ends and having a diameter between 1.4-2.0 mm, and a vertical rod with a diameter between 1.4-2.0 mm,
wherein the distal end has a spherical, hexagonal or flat form between 2.8-3.2 mm in diameter in which firmly tied or fused is a filament of medical grade plastic that is easily visible with a diameter between 0.20-0.35 mm and a length between 10-40 cm to help with placing the device “in situ”, as well as in its removal;
wherein the intrauterine device comprises in the horizontal arms a winding filament or copper wire between 0.24-0.26 mm in diameter and/or a copper tube between 1.5-2.2 mm of outer diameter, of which, the wire or the tube cover the horizontal arms partially or totally;
wherein the vertical rod of the intrauterine device has a winding filament or copper wire between 0.24-0.26 mm of diameter, whose winding covers partly or totally the vertical rod, except the spherical, hexagonal or flat form of the vertical rod;
wherein the copper filament or wire, as well as the copper tube has a purity no less than 99.99%;
wherein the copper area that contains the intrauterine device in form of a “T” will be the sum of the areas developed in the horizontal arms plus the area developed in the vertical rod, and the sum of the areas will be in the range of 200 to 380 mm² of copper with a purity of 99.99% already described.

2. The intrauterine contraceptive device according with claim 1, wherein the device is designed for the Mexican women according to the measurements of the uterine cavity according to its parity characterized by 5 flexible plastic frames in form of a “T” each one with the following measurements:

| WIDTH | | | | LENGTH | | | |
|---|---|---|---|---|---|---|---|
| Parity | Min.* | average | Max.** | Parity | Mini.* | average | Max.** |
| Nulligest 00 | 13 mm | 18 mm | 23 mm | Nulligest 00 | 21 mm | 26 mm | 31 mm |
| Nulliparous 0 | 17 mm | 22 mm | 27 mm | Nulliparous 0 | 23 mm | 28 mm | 33 mm |
| for 1 | 19 mm | 24 mm | 29 mm | for 1 | 25 mm | 30 mm | 35 mm |
| for 2 | 21 mm | 26 mm | 31 mm | for 2 | 27 mm | 32 mm | 37 mm |
| for 3 | 23 mm | 28 mm | 33 mm | for 3 | 29 mm | 34 mm | 39 mm |

*less 5 mm
**less 5 mm

3. The intrauterine contraceptive device according with claim 2, wherein the plastic frame for parity 00 (nulligests or never having been pregnant) has the following measurements width of horizontal arms between 13 mm to 23 mm, average of 18 mm, length of the vertical rod that determines including the diameter of the horizontal arms (without the wire or tube of copper) until the distal end including the spherical, hexagonal or flat form of the device of 21 mm to 31 mm average 26 mm, with a surface of between (200-380) mm² of copper, of 99.99% purity distributed in the horizontal arms and the vertical rod.

4. The intrauterine contraceptive device according to claim 2, wherein the plastic frame for parity 0 (who have not given birth previously) with the following measurements: width of the horizontal arms between 17 mm to 27 mm, average of 22 mm, length of the post or vertical rod that determines including the diameter of the horizontal arms (without the wire or tube of copper) until the distal end including the spherical, hexagonal or flat form of the intrauterine device of 23 mm to 33 mm average 28 mm, with a surface of between (200-380) mm² of copper, of 99.99% of purity distributed in the horizontal arms and of the vertical rod.

5. The intrauterine contraceptive device according with claim 2, wherein the plastic frame for the parity 1 (who have given birth) with the following measurements: width of the tilde or horizontal arms of 19 mm to 29 mm, average of 24 mm, length of the post or vertical rod that determines including the diameter of the tilde or horizontal arms (without the wire or tube of copper) until the distal end including the spherical, hexagonal or flat form of the intrauterine device of 25 mm to 35 mm average 30 mm, with a surface of between (200-380) mm² of copper, of 99.99% purity distributed in the horizontal arms and of the vertical rod.

6. The intrauterine contraceptive device according to claim 2, wherein the plastic frame for parity 2 (with two previous childbirths) with the following measurements: width of the horizontal arms of 21 mm to 31 mm, average of 26 mm, length of the vertical rod which is determined by including the diameter of the horizontal arms (without the wire or copper tube) up to the distal end including the spherical, hexagonal or flat form of the intrauterine device of 27 mm to 37 mm average 32 mm, with a surface of between 200-380 mm² of copper of 99.99% purity distributed in the horizontal arms and in the vertical rod.

7. The intrauterine contraceptive device according to claim 2, whose plastic frame for parity 3 (three previous childbirths) with the following measurements: width of the horizontal arms of 23 mm to 33 mm, average of 28 mm, length of the vertical rod which is determined by including the diameter of the horizontal arms (without the wire or copper tube) until the distal end, including the spherical, hexagonal or flat form of the intrauterine device of 29 mm to 39 mm average 34 mm, with a surface of between (200-380) mm² of copper of 99.99% purity distributed in the horizontal arms and in the vertical rod.

8. The intrauterine contraceptive device according to claim 1, wherein the flexible plastic frame contains a wound in at least one of the horizontal arms, a copper filament between a 0.24 to 0.26 mm of diameter or a copper tube of 2.1 to 2.2 mm of outer diameter and a length of 4.9 to 5.2 mm with a purity of 99.99%, wherein the vertical rod contains a wound a copper filament of between 0.24 and 0.26 mm of diameter whose total surface, of the horizontal arms and the vertical rod from 200 to 380 mm².

9. The intrauterine contraceptive device according to claim 1, further comprising a radiopacifiant material—barium sulphate—, with a proportion in weight of 23±5% and 77±5% of flexible medical grade plastic.

10. The intrauterine contraceptive device according to claim 1, wherein the medical grade plastic has a visco-elastic property or deformation not superior to 5 mm of its original form.

11. The intrauterine contraceptive device according to claim 1, wherein the filament of medical grade plastic has a length between 10 and 40 cm and is tied or fused in the distal end of the vertical rod that has a spherical, hexagonal or flat form.

12. The intrauterine contraceptive device according to claim 1, wherein the T-shaped plastic frame that at the inferior end of the vertical post has an irregular or spherical hexagonal form in which the filament of medical grade plastic is tied or fused in.
